(19) United States
(12) Patent Application Publication
(10) Pub. No.: US 2004/0197834 A1
(43) Pub. Date: Oct. 7, 2004

(54) Method to increase expression of pgd2 receptors and assays for identifying modulators of prostaglandin d2 receptors

(76) Inventors: Francois Gervais, Pierrefonds (CA); Kathleen Mary Metters, Montreal (CA); Gary O'Neill, Ormeaux (CA)

Correspondence Address: MERCK AND CO INC, P O BOX 2000, RAHWAY, NJ, 070650907

(21) Appl. No.: 10/484,363

(22) PCT Filed: Jul. 10, 2002
(86) PCT No.: PCT/CA02/01107

Related U.S. Application Data
(60) Provisional application No. 60/344,519, filed on Nov. 9, 2001.
(60) Provisional application No. 60/306,733, filed on Jul. 20, 2001.

(51) Int. Cl. G01N 33/53; G01N 33/567

(52) U.S. Cl. 435/7.2

(57) ABSTRACT

The present invention provides cell lines expressing endogenous PGD₂-specific receptors, methods for increasing expression of the receptors and assays utilizing the hereindisclosed cell lines for identifying modulators of the PGD₂-specific receptors. Increasing expression of PGD₂-specific receptors is achieved by treating the disclosed cell lines with an agent that induces differentiation.

### CROSS-REFERENCE TO RELATED APPLICATIONS

Not Applicable

### STATEMENT REGARDING FEDERALLY-SPONSORED R&D

Not Applicable

### REFERENCE TO MICROFICHE APPENDIX

Not Applicable

### BACKGROUND OF THE INVENTION

Prostaglandins play physiological and therapeutic roles in human health and various disease states. Prostaglandins not only play a central role in inflammation, but also regulate other critical physiological responses including blood clotting, ovulation, initiation of labor, bone metabolism, nerve growth and development, wound healing, kidney function, blood vessel tone, muscle contraction and relaxation, pain response and immune responses. See Cryer, B., and Feldman, M., Arch Intern. Med. 152:1145-1155, 1992; Whelton, A., and Hamilton, C. W., J. Clin. Pharmacol. 31:588-598, 1994.

Prostaglandin D₂ (PGD₂) is formed in different tissues including brain, spleen, lung, bone marrow, stomach, skin, and also in mast cells. PGD₂ acts through pharmacologically distinct receptor subtypes to modulate cAMP formation and mobilization of intracellular calcium. See Boie, et al., The Journal of Biological Chemistry, 270:18910-18916, 1995 and Hirai, et al., J. Exp. Med., 193:255-261, 2001.

Prostaglandins elicit a diverse spectrum of often opposing biological effects. In humans, prostaglandins are involved in diverse functions, including blood clotting, ovulation, initiation of labor, bone metabolism, nerve growth and development, wound healing, kidney function, blood vessel tone, muscle contraction and relaxation and immune responses. See Cryer, B., and Feldman, M., Arch Intern. Med 152:1145-1155, 1992; Whelton, A., and Hamilton, C. W., J. Clin. Pharmacol. 31:588-598, 1994.

In 1977 investigators demonstrated that Prostaglandin D₂ (PGD₂) was produced by human platelets during aggregation in sufficient quantities to have potential anti-aggregatory effects. Indeed PGD₂ has been shown to inhibit platelet aggregation induced by a number of stimulants such as ADP, collagen, thrombin and arachidonic acid. This inhibition is hypothesized to be closely linked to a stimulation of adenylate cyclase and subsequent cAMP accumulation. It has been suggested that PGD₂ could act as a feedback inhibitor of platelet aggregation and provide a mechanism for preventing aggregation extending inappropriately. Assuming a physiological role for PGD₂ as a feedback inhibitor of aggregation, a decrease in responsiveness to PGD₂ could result in enhanced susceptibility to the thrombus formation associated with each of these conditions. For a review, see Samuelsson B. (ed) Prostaglandins and Related Compounds; Advances in Prostaglandin, Thromboxane, and Leukotriene Research, 21 (A-B), Raven Press, New York (1990).

PGD₂ is formed in a variety of tissues including brain, spleen, lung, bone marrow, stomach, skin, and also in mast cells. PGD₂ has been implicated in many physiological events both in the central nervous system and peripheral tissues.

Within the central nervous system, PGD₂ has been associated with sleep induction, modulation of body temperature, olfactory function, hormone release, nociception and neuromodulation.

Peripherally, PGD₂ has been shown to mediate smooth muscle contraction and relaxation, vasodilation, glycogenolysis, bronchoconstriction and vasoconstriction. For a review, see Giles, et al., Prostaglandins, 35:277-300, 1988. PGD₂ has been implicated in different physiological events such as sleep, pain and inflammation, and allergic responses. (Boie, supra, Narumiya, et al. Physiological Reviews, 79:1193-1226, 1999; Matsuoka, et al. Science, 287:2013-2017, 2000).

The broad range of biological functions attending activation of the prostaglandin signaling pathway via PGD₂ in a number of tissues result from the binding of PGD₂ to a PGD₂ receptor. The physiological and pathophysiological actions of PGD₂ are mediated through interaction with either the DP receptor, the chemoattractant receptor-homologous molecule expressed on TH₂ cell—CRTH₂ or the prostaglandin F2α (FP).

Abramovitz, et al., (U.S. Pat. No. 5,958,723) and Boie (supra), describe the cloning and characterization of the human DP receptor. DP receptor has been shown to be localized in retina, small intestine, platelets, non-chromaffin cells from adrenal medulla, smooth muscle cells from several tissues and nerve tissue, including the central nervous system. Activation of the DP receptor, leads to the stimulation of adenylyl cyclase and an increase in intracellular cAMP levels (Boie supra and Narumiya, supra, 1999).

Abe, et al. Gene, 227:71-77, 1999, Nagata, et al. FEBS Letters, 459:195-199, 1999, and Nagata, et al., The Journal of Immunology, 162:1278-1286, 1999, describe CRTH₂ and its expression on different cells including human T-helper cells, basophils, and eosinophils. Hirai, supra, identify CRTH2 as a receptor for PGD₂ having equal affinity for PGD₂ as the prostanoid DP receptor. Activation of CRTH2 by PGD₂, can lead to an increase in intracellular calcium mobilization (Hirai, supra).

From the very extensive prior art of prostaglandins it is known that this class of substances because of its biological and pharmacological properties is suitable for treating mammals, including man. As such, considerable efforts have been made for identification of chemical compounds that can interrupt these signaling events as potential anti-inflammatory agents.

Thus, selective PGD₂ ligands, agonists or antagonists, depending on which PGD₂ receptor subtype is being considered, would have beneficial anti-inflammatory and analgesic properties due to blockade of inflammatory and pain-mediator production.

However, development of therapeutic compounds that modulate PGD₂ receptor or ligand related activity including the ensuing second messenger cascade require a sensitive assay, which is currently unavailable, especially those in which the cells produce endogenous native PGD₂ receptors.

Indeed, a chief drawback attending such conventional assays is the low level of expression of receptors activated by PGD₂. Indeed, the level of expression of receptors activated by PGD₂ in the aforementioned prior art cells varies from medium to low, which effectively lowers the overall sensitivity of the assay.

Thus, there is increasing emphasis on increasing level of PGD₂ expression in cells that endogenously produce native receptors and the use of such cell lines or cell membranes containing said receptors in the development of assays for identifying specific compounds that inhibit the activation of receptors by PGD₂ and thus inhibit the signaling event attending the prostaglandin pathway as well as evaluating the activity of any compound thus identified as an anti-inflammatory agent. The proposed assay can also be used to evaluate the effectiveness of potential anti-inflammatory agents, at the molecular level, as well as for reagents for use in such methods.

Consequently, the present invention aims to overcome the disadvantages attending conventional cell lines and assays utilizing said cell lines by identifying two specific cell lines that endogenously express PGD₂ receptors capable of being activated by a PGD₂ receptor binding ligand which differs pharmacologically from previously identified receptors. The proposed assay makes use of the disclosed cell lines and will facilitate identification of PGD₂ receptor modulators including agonists and antagonists. Some compounds identified therewith will have therapeutic utility.

### OBJECTS AND SUMMARY OF THE INVENTION

The present invention relates to cell lines capable of endogenously expressing PGD₂-specific receptors and makes use of these cell lines to assay for a compound able to modulate the activity of PGD₂-specific receptors. Activities mediated by PGD₂-specific receptors include changes in second messenger activity such as cAMP, inositol phosphate, mobilization of calcium ions, degranulation, chemokinetic and chemotactic effects. Compounds modulating PGD₂ receptor activity include agonists, antagonists and allosteric modulators. Determining the ability of a compound to modulate PGD₂ receptor activity can be achieved quantitatively or qualitatively.

More particularly, the invention includes cell lines that endogenously express the PGD₂ specific receptors, CRTH2 and DP. Examples of such cell lines are AML14.3D10 and HL60.

Another aspect of the invention is a method of inducing cell differentiation comprising treating a cell with an agent in an amount sufficient to induce cellular differentiation thereby increasing the expression of a prostaglandin D₂ (PGD₂) receptor, wherein the inducing agent is selected form the group consisting of butyric acid, dimethyl sulfoxide, IL-5, retinoic acid, dibutyryl cyclic-AMP and 5-bromodeoxyuridine.

In another aspect, the invention provides assays using the disclosed cell lines or their plasma membrane to screen for a test compound that interacts with or modulates the activity of PGD₂-specific receptors. The assays employ either of the two disclosed cell lines.

Another aspect of the present invention describes a method of screening for a test compound that modulates the biological and/or pharmacological activity of a prostaglandin D₂ receptor comprising:

(a) contacting a cell expressing a PGD₂-specific receptor with a test compound,

(b) adding a PGD₂ or a PGD₂ analogue to the cells,

(c) measuring an effect of the test compound on either an intracellular second messenger or chemokinesis as a measure of the ability of the test compound to modulate PGD₂-specific receptor activity,

(d) repeating (a) to (c) in the absence of the test compound, and

(e) comparing the results of (c) and (d).

In accordance with this aspect, the invention features a method of detecting an agonist or antagonist of PGD₂ receptor comprising the steps of incubating cells that produce an endogenous receptor activated by PGD₂ or a PGD₂ analogue in the presence and absence of a test compound, and detecting changes in the level of PGD₂ related activity, including measuring the level of increase or decrease of second messenger activity such as cAMP and Ca⁺⁺; degranulation; chemokinetic and chemotactic effects.

In another aspect, the present invention describes a method of screening for a test compound that binds to a prostaglandin D₂ receptor comprising:

(a) incubating a labeled PGD₂ or PGD₂ analogue and a test compound with a cell expressing a PGD₂-specific receptor or a plasma membrane therefrom, allowing the labeled PGD₂ or PGD₂ analogue to compete with the test compound and bind to the cell expressing PGD₂-specific receptor or the plasma membrane therefrom,

(b) removing the unbound labeled PGD₂ or PGD₂ analogue,

(c) measuring the amount of the bound labeled PGD₂ or PGD₂ analogue as a measure of the ability of the test compound to compete with the labeled PGD₂ or PGD₂ analogue for the PGD₂-specific receptor, and

(d) comparing the results in the absence of the test compound.

Another aspect of the invention is drawn to a pharmaceutical composition comprising a PGD₂ receptor agonist or antagonist, selected using the herein disclosed assays, in an amount sufficient to alter PGD₂ associated activity, and a pharmaceutically acceptable diluent, carrier, or excipient.

At least some of these and other objects are addressed by the various embodiments of the invention disclosed herein. The provided examples illustrate different components and methodology useful in practicing the present invention. The examples do not limit the claimed invention. Based on the present disclosure the skilled artisan can identify and employ other components and methodology useful for practicing the present invention. Other features and advantages of the invention will be apparent to those of skill in the art upon further study of the specification and claims.

### BRIEF DESCRIPTION OF THE FIGURES

FIG. 1, shows the results of a binding assay i.e., specific binding of labeled PGD₂ to membranes.

FIG. 2, shows results of the specific binding of radioactive PGD₂ to cell membranes derived from butyric acid-differentiated HL-60 or undifferentiated AML14.3D10 cells.

### DETAILED DESCRIPTION OF THE INVENTION

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described.

All publications mentioned herein are incorporated herein by reference as if each individual publication were specifically and individually indicated to be incorporated by reference herein as though fully set forth. Nothing herein is to be construed as an admission that the invention is not entitled to antedate such disclosure by virtue of prior invention. All publications, including but not limited to patents and patent applications, cited in this specification are herein incorporated by reference.

In the description that follows, a number of terms used in the field of recombinant DNA technology are extensively utilized. In order to provide a clearer and consistent understanding of the specification and claims, including the scope to be given such terms, the following definitions are provided.

As a first aspect, provided herein are cell lines that endogenously express PGD₂ receptors. These receptors bind PGD₂ and PGD₂ analogues. These cell lines and cell membranes derived therefrom (plasma membranes) can be used in assays to screen for compounds that modulate PGD₂ receptors such as allosteric modulators, agonists and antagonists. Particularly, these assays are useful in identifying compounds that interact specifically With PGD₂ specific receptors.

A distinguishing feature attending the present invention is the discovery that the herein disclosed cell lines have been found to express at least two PGD₂ specific receptors on their surface. These are the DP and CRTH2 receptors.

The DP receptor is coupled to the G-protein Gas, which when activated, is an effector of biological processes, including the stimulation of adenylate cyclase and phospholipases. Activation of the DP receptor by PGD₂ or a PGD₂ analogue, allows the Gαs protein to stimulate adenylate cyclase which leads to the production of cAMP. Further, the stimulation of phospholipases initiates inositol phosphate (IP) release which affects Ca⁺⁺ mobilization. Therefore, contacting cells expressing a DP receptor with agonists will cause increase of cAMP accumulation and/or result in a change in Ca⁺⁺ mobilization, antagonists will block this effect.

The CRTH2 receptor is coupled to the G-protein Gαi, which when activated is an effector to biological processes, including the inhibition of adenylate cyclase and activation of phospholipases (Hirai, supra). Activation of the CRTH2 receptor by PGD₂ or a PGD₂ analogue, activates the Gαi protein which results in the inhibition of adenylate cyclase. To evaluate the level of adenylate cyclase inhibition in cells, the cells are first treated with an agent to induce the accumulation of cAMP (an example of such an agent is forskolin). Therefore, treating cells expressing CRTH2 receptor with an agent such as forskolin, thereby inducing the accumulation of cAMP and then contacting these same cells with an agonist, results in a decrease in the accumulation of forskoln-induced cAMP accumulation. Antagonists to CRTH2 block this effect. A further indication of modulating the CRTH2 is as recited above, the effect on phospholipases affects the release of IP which in turn affects Ca⁺⁺ mobilization.

The activation of the PGD₂ receptor by PGD₂ or PGD₂ analogue, leads to the stimulation of phospholipase C or D which liberates inositol phosphate. Thus, another aspect of the invention relates to an assay to identify compounds that modulate the activity of the activated PGD₂ receptor by measuring IP or a downstream marker such as Ca⁺⁺ mobilization.

Assays using the cell lines of the present invention are useful in screening for compounds that modulate PGD₂ receptor activity. Beneficial effects of modulating PGD₂ receptor activity include achieving one or more of the following in a patient: treatment or prevention of an inflammatory disease such as asthma or arthritis; treatment or prevention of allergic rhinitis and treatment or prevention of a sleep disorder. A patient is a mammal, preferably a human. The term patient includes subjects treated prophylactically, preventive and subjects afflicted with a disease or disorder.

Selective agonists or antagonists that mimic or block PGD₂ actions at the DP receptor and/or CRTH2 receptor may have utility in the treatment of disease states or diseases not limited to allergic rhinitis, and other allergic conditions in which mast cells, eosinophils, TH2 cells and other immune cells express the DP receptor and/or CRTH2 receptor or produce PGD₂. Additional examples of therapeutic applications include one or more of the following: sleep disorder; glaucoma; osteoporosis; modulators may be useful as cytoprotective, analgesic or anti-inflammatory agents; modulators inhibiting platelet aggregation may be useful in treating vascular diseases, prevention of post-injury blood clotting, rejection in organ transplant and by-pass surgery, congestive heart failure, pulmonary hypotension and Raynaud's disease.

The “AML 14” cell line is a leukemic cell line, which is blast-like but exhibits characteristics of eosinophils after stimulation with cytolines. It has been shown to be an effective model for the study of the regulation of the growth of eosinophils. A stable cloned subline AML 14.3D10 that spontaneously exhibits characteristics of eosinophils in the absence of cytokine differentiation has been established. See Paul, C. C. et al., Blood, 86 10:3737-3744, 1995.

“Undifferentiated” refers to cells that have not been treated with an agent to induce or enhance differentiation. “Differentiated” refers to cells that have been treated with an agent capable of inducing or enhancing differentiation. A differentiated cell, is a cell having specific biological and functional characteristics. Examples of differentiating agents include butyric acid, IL-5, 5-bromodeoxyuridine, dibutyryl-cAMP, retinoic acid and dimethyl sulfoxide. For additional examples of differentiating agents see Journal of Leukocyte Biology, 56:74, 1994 and Scogan et. al. Eur. J. Biochem., 239:572-578, 1996. Inducing or enhancing differentiation in a cell can be conducted using one or a mixture of two or more differentiating agents. A non-limiting example is a mixture of butyric acid and IL-5.

While the AML 14.3D10 and HL60 cell lines are known in the art, a characteristic feature of the present invention is the discovery that these cell lines endogenously express receptors that bind PGD₂ and PGD₂ analogues. Advantageously, these cell lines, when treated to induce differentiation demonstrate increased cell surface expression of PGD₂ receptors.

Therefore, a novel discovery of the presence of these receptors in the cell lines and the increased cell surface expression of these receptors upon inducing differentiation, provide a means for one skilled in the art to utilize the herein disclosed cell lines in assays to screen for modulators of PGD₂ receptor activity and identify potential agonists and/or antagonists of PGD₂ or its receptors. Compounds identified using the disclosed cell lines can be used to treat PGD₂ mediated conditions.

Both non-differentiated and differentiated AML 14.3D10 cells and differentiated HL60 cells are useful in screening for compounds capable of modulating and/or binding the receptors expressed by these cells.

A particular aspect of the present invention that is of interest is the induction of cellular differentiation in the cell lines AML 14.3D10 and HL60, thereby increasing or enhancing cell surface expression of PGD₂ receptors. Differentiating agents useful in inducing cellular differentiation include butyric acid, IL-5, 5-bromodeoxyuridine, dibutyryl-cAMP, retinoic acid and dimethyl sulfoxide. Inducing or enhancing differentiation in a cell can be conducted using one or more differentiating agents. Preferably, butyric acid is used alone or with at least one other agent. A preferred combination is butyric acid and IL-5.

Though other PGD₂ receptor type may be expressed on the cell surface of the cell lines disclosed herein, CRTH2 is expressed predominantly and DP is expressed to a lesser amount. Compounds capable of modulating and/or binding PGD₂ cell receptors may be selected by using intact cells or plasma membrane generated from the herein disclosed cell lines. Further, compounds that specifically modulate and/or bind CRTH2 and/or DP receptors may be selected by using intact cells or plasma membrane generated from the herein disclosed cell lines.

The term “PGD₂ analogue” encompasses naturally occurring and synthetic counterparts that are capable of binding to a native PGD₂ receptor, this term is interchangeably used with the term “ligand”.

The term “modulates the activity” of a PGD₂ receptor refers to a change in the activity of a PGD₂ receptor in the presence of a compound when compared to the activity of a PGD₂ receptor in the absence of the compound. Such compounds include allosteric modulators, agonists and antagonists.

The effect of PGD₂ or PGD₂ analogue on PGD₂ receptors expressed by either of the two cell lines disclosed herein in the absence or presence of a test compound can be evaluated using cell-based functional assays. These assays include measuring second messenger activity such as inositol phosphate production (Swann et al., Journal of Cell Biology, 103:2333-2342, 1986), intracellular calcium mobilization and cAMP levels, (Boie, supra); degranulation; chemokinesis and chemotaxis (Gervais et al., J. All. Clin. Imm. in press, 2001).

The term “agonist” refers to a substance or signal that activates receptor function; and the term “antagonist” refers to a substance that interferes with receptor function. Typically, the effect of an antagonist is observed as a blocking of activation by an agonist.

The effectiveness of an agonist to alter PGD₂ receptor activity can be evaluated by comparing PGD₂ receptor activity in the presence and absence of the agonist. Different types of assay formats can be employed. (Boie, supra, 1995 and Hirai, supra, 2001).

The ability of a compound to antagonize PGD₂ receptor activity can be evaluated using a PGD₂ agonist able to produce receptor activity and then measuring the ability of one or more test compounds to alter such activity. Agonists that can be employed include those able to stimulate both DP receptor activity and CRTH2 activity and those selective for DP receptor activity or CRTH2 activity. Examples of different types of agonists are PGD₂ which acts at both the DP receptor and CRTH2; 13-14-dihydro-15-keto-PGD₂ which is specific for CRTH2; and BW245C which is specific for the DP receptor.

The effectiveness of an antagonist to alter PGD₂ receptor activity can be evaluated by comparing PGD₂ receptor activity in the presence of the agonist, and, in the presence and absence of an antagonist. Different assay formats can be employed. For example, a control experiment involving an agonist and, a test experiment involving the agonist and a test compound can be performed at the same or at different times.

One method included herein involves screening for PGD₂ receptor inhibitors by determining inhibition or stimulation of receptor-mediated cAMP accumulation and/or adenylate cyclase activity. Such a method involves incubating or exposing PGD₂ expressing cells to potential antagonists in the presence of an agonist of the PGD₂ receptor and measuring the accumulation of cAMP. If the potential antagonist binds the receptor, and thus inhibits receptor activation by the agonist, the level of receptor-mediated cAMP or adenylate cyclase activity is affected. It should be noted that the type of receptor that binds the potential antagonist determines whether there is an increase or decrease in second messenger activity.

In general, agonists of DP increase cAMP whereas antagonists of DP prevent increases in cAMP mediated by DP agonists. Likewise, agonists of CRTH2 inhibit the forskolin-induced accumulation of cAMP and antagonists of CRTH2 block the effect of CRTH2 agonists. As well, agonists of DP and agonists at CRTH2 both lead to an increase in intracellular calcium, and antagonists of DP and CRTH2 both prevent the intracellular calcium increase mediated by agonists.

Methods for identifying compounds that modulate the biological activity of PGD₂ receptor comprise contacting or exposing cells expressing endogenous PGD₂ cell surface receptor with a PGD₂ or a PGD₂ analogue in the presence or absence of a test compound and measuring an effect of the test compound on biological activity.

Accordingly, an embodiment of the present invention is a method of screening for a test compound capable of modulating prostaglandin D₂ receptor activity comprising:

(a) contacting a cell expressing a PGD₂-specific receptor with a test compound,

(b) adding a PGD₂ or a PGD₂ analogue to (a),

(c) measuring the effect of the test compound on either an intracellular second messenger or chemokinesis, as a measure of the ability of the test compound to modulate PGD₂-specific receptor activity,

(d) repeating (a) to (c) in the absence of the test compound, and

(e) comparing the results of (c) and (d).

In an aspect of this embodiment the PGD₂ receptors are CRTH2 and DP.

In another embodiment of this aspect the cell lines that endogenously express PGD₂ receptors include differentiated and undifferentiated AML 14.3D10, and differentiated HL60.

In an additional aspect of this embodiment, an agent capable of inducing differentiation in AML 14.3D10 and HL60 cell lines include at least one of: butyric acid, IL-5, 5-bromodeoxyuridine, dibutyryl-cAMP, retinoic acid and dimethyl sulfoxide. Preferably, butyric acid is used alone or in combination with at least one other agent. A preferred mixture is butyric acid and IL-5.

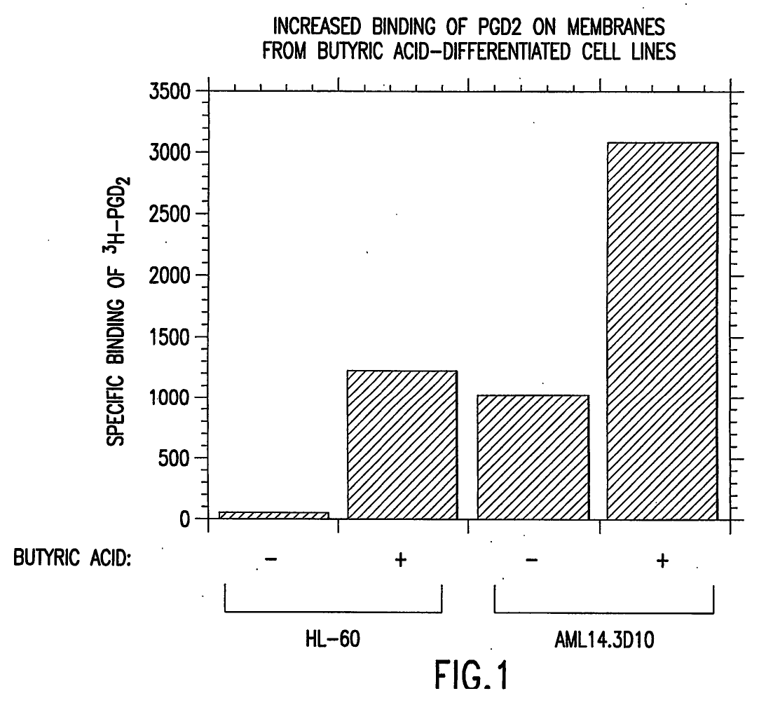

For example, referring to FIG. 1, shown therein are the results of a binding assay, specifically the binding of radioactive PGD₂ to membranes. The assay measures the amount of labeled receptor bound to cell membranes derived from the herein disclosed cell lines. Briefly, AML14.3D10 and BL60 cell lines are incubated in the absence and/or presence of 0.4 nM of butyric acid for 7 days. Membranes were thereafter isolated from the cells and used in aforementioned PGD₂ binding assays.

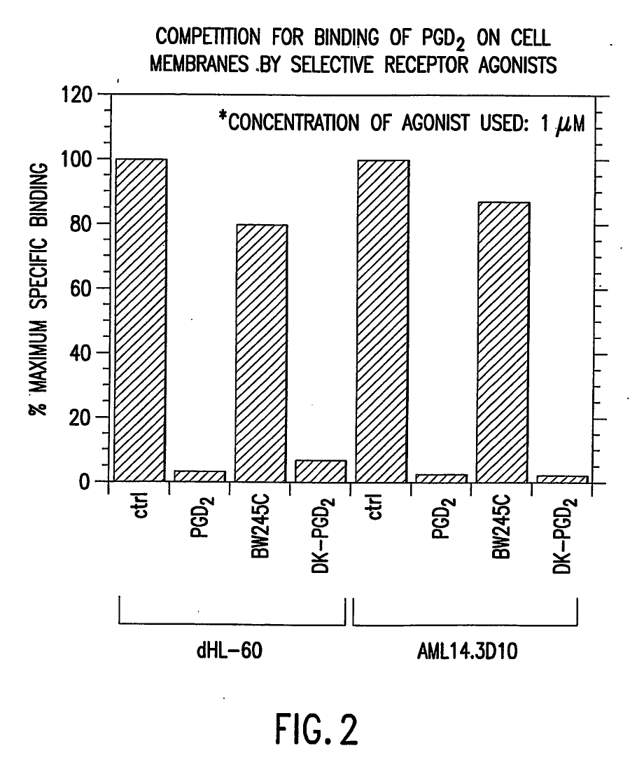

Likewise, FIG. 2, shows results of the specific binding of radioactive PGD₂ to cell membranes derived from butyric acid-differentiated HL-60 or undifferentiated AML14.3D10 cells. Membranes were isolated from butyric acid-differentiated HL-60 or undifferentiated AML14.3D10 and used in the PGD₂ binding assay in the presence or absence of various 1 uM of non-labeled receptor agonists described here below:

ctrl: in the absence of a competitor;

PGD₂: dual DP/CRTH2 agonist;

BW245C: DP-selective agonist;

DK-PGD₂: CRTH2-selective agonist.

Specific binding of radioactive PGD₂ to the membrane was measured and compared to the value obtained in the absence of competitor (ctrl=100% maximum binding).

In a further aspect of this embodiment, the differentiated AML 14.3D10 and HL60 cell lines have increased expression of PGD₂ receptors when compared to cells that have not been differentiated.

In an additional aspect of this embodiment determining the effect on intracellular second messenger includes measuring the amount of cAMP accumulation, IP released and Ca⁺⁺ mobilization. A specific aspect is measuring the amount of cAMP accumulation as a determination of the effect on intracellular second messenger.

Another aspect of the invention is a screening technique which involves the use of a microphysiometer. A microphysiometer is useful in that it allows the detection of the interaction between a receptor and a test compound without the labeling of either the receptor or the test compound. McConnell, H. M. et al. Science, 257:1906-1912, 1992. Briefly, a “microphysiometer” (e.g., Cytosensor) is an analytical instrument that measures the rate at which a cell acidifies its environment using a light-addressable potentiometric sensor (LAPS). Binding between the receptor and the test compound lead to cellular activity, an increase in metabolism and as a consequence generation of acidic metabolites. Thus a change in the acidification rate is an indication of binding between the receptor and the test compound.

In an aspect of this embodiment, the method is scaled up for high throughput screening for a compound capable of modulating PGD₂ receptor activity, wherein a single compound or a mixture of two or more compounds are screened.

The present invention encompasses binding assays for selecting a compound capable of binding the PGD₂ receptor. The binding assays of the present invention include displacement and competition type of binding assays. The binding assays disclosed herein are exemplary, the invention comprises other binding assays known in the art in which the cells of the present invention can be utilized. Further, a person skilled in the art can easily adapt the assays disclosed herein and in the art to conduct high throughput screening (HTS) for selecting a compound capable of binding a PGD₂ receptor, therefore HTS are comprised in the present invention.

Briefly, in the displacement binding assay, PGD₂ or a PGD₂ analogue is labeled and added to the cells expressing PGD₂ receptors or plasma membrane generated therefrom and incubated for a time sufficient to allow binding. Incubations may be performed at any temperature which facilitates optimal binding, typically between about 4 and about 40° C. Typically an incubation period between about 0.1 and about 2 hour will be sufficient. The test compound is then added and the bound labeled component is measured. The amount of bound label measured is an indication of the ability of the test compound to bind to the PGD₂ receptor by displacing the labeled PGD₂ or PGD₂ analogue. Alternatively, the assay is conducted as described except that the test compound is added first to the cells or cell membranes, followed by the addition of a labeled PGD₂ or PGD₂ analogue. The amount of label is an indication of the displacement of the test compound by the labeled PGD₂ or PGD₂ analogue and is a measure of the test compound's ability to remain bound to the PGD₂ receptor.

Additionally the invention encompasses an assay wherein the test compounds are labeled and the PGD₂ or PGD₂ analogue is not labeled.

Accordingly, in another embodiment of the present invention is a method of screening for a test compound that binds to a prostaglandin D₂ receptor comprising:

(a) mixing a labeled PGD₂ or PGD₂ analogue with a cell expressing a PGD₂-specific receptor or to a plasma membrane therefrom, allowing the labeled PGD₂ or PGD₂ analogue to bind to the cell expressing PGD₂-specific receptor or to the plasma membrane therefrom,

(b) contacting a test compound to mixture (a),

(c) removing the unbound labeled PGD₂ or PGD₂ analogue,

(d) measuring the amount of the bound labeled PGD₂ or PGD₂ analogue as a measure of the ability of the test compound to bind to the PGD₂-specific receptor, and

(e) comparing the results in the absence of the test compound.

In an aspect of this embodiment the PGD₂ receptors are CRTH2 and DP.

In another aspect of this embodiment the cell lines that endogenously express PGD₂ receptors include differentiated and undifferentiated AML 14.3D10, and differentiated HL60.

In a further aspect of this embodiment the differentiated AML 14.3D10 and HL60 cell lines have increased expression of PGD₂ receptors when compared to cells that have not been differentiated.

In an additional aspect of this embodiment, an agent capable of inducing differentiation in AML 14.3D10 and HL60 cell lines includes at least one of: butyric acid, IL-5, 5-bromodeoxyuridine, dibutyryl-cAMP, retinoic acid and dimethyl sulfoxide. Preferably, butyric acid is used alone or in combination with at least one other agent. A preferred mixture is butyric acid and IL-5.

The present invention also encompasses a binding assay such that the test compound to be screened is labeled and the PGD₂ or a PGD₂ analogue is not labeled. All other steps in this particular embodiment are the same.

Therefore, in an another aspect of this embodiment, the assay is conducted such that the test compound is labeled and the PGD₂ or PGD₂ analogue is not labeled. Displacement of the labeled test compound by the non-labeled PGD₂ or PGD₂ analogue is an indication of the ability of the test compound bind the PGD₂ receptor.

In an additional aspect of the present embodiment, the means for labeling a compound include radioisotope and fluorescent labeling, a person skilled in the art would know how to label a test compound and PGD₂ or PGD₂ analogue with a radioisotope or a fluorescent compound, and the appropriate means for the detection of the label.

In the competition type binding assay, the labeled PGD₂ or PGD₂ analogue and the test compound are added at the same time to the cell expressing PGD₂-specific receptor or to the plasma membrane therefrom. By maintaining the amount of the labeled PGD₂ or PGD₂ analogue constant and varying the amount of the test compound, the ability of the test compound to compete with PGD₂ or PGD₂ analogue for the PGD₂-specific receptor is determined.

Therefore, in a further embodiment of the present invention is a method of screening for a test compound that binds to a prostaglandin D₂ receptor comprising:

(a) incubating a labeled PGD₂ or PGD₂ analogue and a test compound with a cell expressing a PGD₂-specific receptor or to a plasma membrane therefrom, allowing the labeled PGD₂ or PGD₂ analogue to compete with the test compound to bind to the cell expressing PGD₂-specific receptor or to the plasma membrane therefrom,

(b) removing the unbound labeled PGD₂ or PGD₂ analogue,

(c) measuring the amount of the bound labeled PGD₂ or PGD₂ analogue, as a measure of the ability of the test compound to compete with the labeled PGD₂ or PGD₂ analogue for the PGD₂-specific receptor, and

(d) comparing the results in the absence of a test compound.

In an aspect of this embodiment the PGD₂ receptors are CRTH2 and DP.

In another aspect of this embodiment the cell lines that endogenously express PGD₂ receptors include differentiated and undifferentiated AML 14.3D10, and differentiated HL60.

In a further aspect of this embodiment the differentiated AML 14.3D10 and HL60 cell lines, have increased expression of PGD₂ receptors when compared to cells that have not been differentiated.

In an additional aspect of this embodiment, an agent capable of inducing differentiation in AML 14.3D10 and HL60 cell lines include at least one of: butyric acid, IL-5, 5-bromodeoxyuridine, dibutyryl-cAMP, retinoic acid and dimethyl sulfoxide. Preferably, butyric acid is used alone or in combination with at least one other agent. A preferred mixture, is butyric acid and IL-5.

In an additional aspect of the present embodiment, the means for labeling a compound include radioisotope (such as ¹²⁵Iodine or ³H) and fluorescent labeling, a person skilled in the art would know how to label a test compound and PGD₂ or PGD₂ analogue with a radioisotope or a fluorescent compound, and the appropriate means for the detection of the label.

In an aspect of the present invention, is a method to determine the ability of the PGD₂ receptor to interact with its target molecule using a technology such as real-time Biomolecular Interaction Analysis (BIA). Sjolander, S. and Urbaniczky, C., Anal. Chem., 63:2338-2345, 1991. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

Agonists and/or antagonists may be identified from a variety of sources, for instance, from cells, cell-free preparations, chemical libraries and natural product mixtures. Such agonists and/or antagonists may be natural or modified substrates, ligands, receptors, enzymes, etc., as the case may be, of PGD₂ or PGD₂ analogue; or may be structural or functional mimetics, of the PGD₂ or PGD₂ analogue. See Coligan et al. Current Protocols in Immunology, 1(2):Chapter 5, 1991. Potential PGD₂ receptor antagonists may include an antibody which binds to the G-protein coupled receptor, but does not induce a second messenger response, such that the activity of the G-protein coupled receptor is prevented. Other potential antagonists include small molecules, e.g. small peptides or peptide-like molecules or organic molecules, which bind to the PGD₂ receptor, making it inaccessible to ligands, such that normal biological activity is prevented. Potential antagonists may also include molecules that are closely related to the ligand of the PGD₂ receptor, i.e. a fragment of the ligand, which have lost biological function, and which, when binding to the PGD₂ receptor, elicit no response.

Another potential antagonist is a soluble form of the PGD₂ receptor, for example a fragment of the receptor, which binds to PGD₂ and prevents PGD₂ from interacting with the membrane bound PGD₂ receptor.

Compounds identified using the cell lines and assays of the present invention can be of therapeutic applications. Accordingly, in a further aspect, the present invention provides a compound identified as an agonist or an antagonist of the PGD₂ receptor for therapeutic applications.

Agonists and antagonists for PGD₂ receptors may be used for therapeutic purposes, such as treatment for various disease states in which PGD₂ or its receptor play a role such as for example the treatment for rheumatoid arthritis and other inflammatory diseases; asthma; sleep disorder; glaucoma; osteoporosis; analgesia; inhibitors of platelet aggregation may be useful in treating vascular disease such as atherosclerosis; prevention of post-injury and post-operative blood clotting; rejection in organ transplant and by-pass surgery; congestive heart failure; pulmonary hypotension and Raynaud's disease.

Accordingly, in a further aspect, this invention provides a method of treating an abnormal condition related to an excess of PGD₂ receptor activity and/or a ligand thereof, for example PGD₂, which comprises administering to a patient in need thereof an antagonist as hereinbefore described in an amount effective to block binding of ligands to the receptor, and thereby alleviating the abnormal condition.

This invention also provides a method of treating an abnormal condition related to an under-expression of PGD₂ receptor activity and/or a ligand thereof which comprises administering to a patient in need thereof a therapeutically effective amount of an agonist compound which activates the receptor as hereinbefore described and thereby alleviates the abnormal condition.

Accordingly, in a further aspect, the present invention provides for, a pharmaceutical composition comprising a compound identified as an inhibitor or an activator of the PGD₂ receptor and a pharmaceutically acceptable excipient or carrier. The PGD₂ agonists or antagonists may be administered in combination with a suitable pharmaceutical carrier e.g. saline, buffered saline, dextrose, water, glycerol, ethanol, or combinations of these agents. The formulation is comprised of a therapeutically effective amount of the agonist or antagonist in combination with a pharmaceutically acceptable carrier. The formulations may be administered by topical, intravenous, intraperitoneal, intramuscular, intranasal or intradermal routes, in amounts which are effective for treating and/or prophylaxis of the specific indication. In general, the formulations will be administered in an amount of at least 10 μg/kg body weight to as high as about 8 mg/kg body weight. In most cases, the dosage is from about 10 μg/kg to about 1 mg/kg body weight daily, depending on the route of administration, symptoms and severity of the disease condition. Dosages can be determined based upon the effectiveness of treatment of the underlying disease state, side effect profile and other factors.

Compounds which are active when given orally can be formulated as liquids, for example syrups, suspensions or emulsions, tablets, capsules and lozenges.

A liquid formulation will generally consist of a suspension or solution of the compound or pharmaceutically acceptable salt in a suitable liquid for example, water, ethanol, glycerine, or in non-aqueous solvent, for example polyethylene glycol, or oils.

A composition in the form of a tablet can be prepared using any suitable pharmaceutical carrier(s) routinely used for preparing solid formulations. Examples of such compounds include magnesium stearate, starch, lactose, sucrose and the like.

A composition in the form of a capsule can be prepared using routine encapsulation procedures. For example, pellets containing the active ingredient can be prepared using standard carriers and then filled into a hard gelatin capsule; alternatively, a dispersion or suspension can be prepared using any suitable pharmaceutical carrier(s), for example aqueous gums, celluloses, silicates or oils and the dispersion or suspension then filled into a soft gelatin capsule.

Typical parenteral compositions consist of a solution or suspension of the compound or pharmaceutically acceptable salt in a sterile aqueous carrier or parenterally acceptable oil, for example polyethylene glycol, polyvinyl pyrrolidone, lecithin, arachis oil or sesame oil.

Alternatively, the solution can be lyophilized and then reconstituted with a suitable solvent just prior to administration.

A typical suppository formulation comprises an active compound or a pharmaceutically acceptable salt thereof which is active when administered in this way, with a binding and/or lubricating agent such as polymeric glycols, gelatins or cocoa butter or other low melting vegetable or synthetic waxes or fats.

Preferably the composition is in unit dose form such as a tablet or capsule.

It may be desirable to immobilize either the PGD₂ receptor or its target molecule to facilitate separation of complexed from non-complexed forms of the proteins, as well as to accommodate automation of the assay. Interaction of a PGD₂ receptor with a target molecule in the presence and absence of a test compound, can be accomplished in any vessel suitable for containing the reactants e.g., microtitre plates, test tubes and micro-centrifuge tubes. In one embodiment, a fusion protein is provided which adds a domain that allows one or both of the proteins to be bound to a matrix. For example, glutathione-S-transferase/PGD₂ receptor fusion proteins or glutathione-S-transferase/target fusion proteins can be adsorbed onto glutathione sepharose beads (Sigma Chemical, St. Louis, Mo.) or glutathione derivatized microtitre plates, which are then combined with the test compound and either the non-adsorbed target protein or PGD₂ receptor, and the mixture incubated under conditions conducive to complex formation (e.g., at physiological conditions for salt and pH). Following incubation, the beads or microtitre plate wells are washed to remove any unbound components; the matrix is immobilized in the case of beads and the complex is determined either directly or indirectly. Alternatively, the complexes can be dissociated from the matrix and the level of PGD₂ receptor binding activity determined using standard techniques.

Preferably, the PGD₂ receptor preparation is derived from either the AML14.3D10 cell line or the differentiated AML14.3D10 and HL 60 cell line.

The components useful to practice the assays of the present invention can be contained in kits. One such kit for the binding assay would include the cell lines of the present invention or plasma membrane therefrom and a differentiating agent, e.g., butyric acid, appropriate buffers, an adenylate cyclase activator, e.g., forskolin, labeled and non-labeled components and positive and negative controls.

The examples below are carried out using standard techniques, which are well known and routine to those of skill in the art, except where otherwise described in detail. The following examples illustrate, but do not limit the claimed invention.

### EXAMPLE 1

Cells

HL-60 cells were obtained from ATCC as culture CRL-1964. AML14.3D10 were generated by Dr. Cassandra Paul (licensed by Merck). Cells are grown in a humidified atmosphere at 37° C. (6% CO₂) in RPMI-1640 media supplemented with 10% fetal calf serum, 2 mM glutamine, 100 U/ml penicillin-G and 100 μg/ml streptomycin (obtained from GIBCO-BRL).

Differentiation of HL60 Cells and AML14.3D10 Cells

On the first day of the experiment cells are split at 2×10⁵ cells/ml and supplemented with 0.4 mM butyric acid. On the fourth day the cells are diluted to 2×10⁵ cells/ml and the concentration of butyric acid is adjusted to 0.4 mM. The cells are harvested on the seventh day.

### EXAMPLE 2

Preparation of Membranes

HL-60 or AML14.3D10 cells are collected by centrifugation for 6 min at 300 g at 4° C., washed with PBS, centrifuged as before and suspended in 10 mM HEPES/KOH pH 7.4, 1 mM EDTA. The cells are disrupted by nitrogen cavitation (800 psi for 30 min. on ice) in the presence of protease inhibitors (2 mM AEBSF, 10 μM E-64, 100 μM leupeptin and 0.05 mg/ml pepstatin). Cell membrane is isolated by differential centrifugation at 4° C., first at 1000 g for 10 min then 160 000 g for 30 min. After centrifugation the pellet comprising the cell membrane is suspended in 10 mM HEPES/KOH pH 7.4, 1 mM EDTA, pH 7.4 buffer using Dounce homogenization.

### EXAMPLE 3

Expression of CRTH2 and DP Receptors in HL-60 and AML14.3D10 Cells

There is a minimal amount of PGD₂ binding on the membrane prepared from undifferentiated HL-60 cells and an appreciable level of PGD₂ binding on membranes from undifferentiated AML14.3D10 cells as determined by radioligand binding assay. With butyric acid induced differentiation of cell lines HL-60 and AML14.3D10, there is a significant increase in PGD₂ binding at the PGD₂ receptors. About 90% of this differentiation-induced increase in PGD₂ binding is competed away, in both HL-60 and AML14.3D10, by the presence of the CRTH2 selective ligand, DK-PGD₂ (obtained from Cayman Chemical, Ann Arbor, Mich.). The remaining about 10% is competed away by the presence of the DP selective ligand, BW245C (obtained from Cayman Chemical, Ann Arbor, Mich.). This data demonstrates that the increase in PGD₂ binding on differentiated HL-60 and AML14.3D10 is mostly due to increased expression of CRTH2 receptor (unpublished data). PGD₂ is obtained from Biomol Research laboratories (Plymouth Meeting, Pa.).

### EXAMPLE 4

Binding Assay

Ligand binding assays provide direct methods for ascertaining receptor pharmacology and are adaptable to a high throughput format. The purified ligand for a receptor is radiolabeled to high specific activity (50-2000 Ci/mmol). A determination is made that the process of radiolabeling does not diminish the activity of the ligand towards its receptor. Assay conditions such as buffers, ions, pH and incubation time are optimized to establish a workable signal to noise ratio for both membrane and whole cell receptor assays. Such conditions are well known to one skilled in the art.

For these assays, specific receptor binding is defined as total associated radioactivity minus the radioactivity measured in the presence of an excess of unlabeled competing ligand. Where possible, more than one competing ligand is used to define residual nonspecific binding. Ligand binding assays are performed using whole cells or cell membranes derived therefrom.

a. Whole Cell Assay

³H-labeled PGD₂ is incubated with a predetermined number of one of the two disclosed cell lines in the presence or absence of increasing concentration of a test compound. The incubation is carried out at room temperature. Following the incubation, the cells are washed, collected and the radioactivity counted (Hirai, supra).

Alternatively, the assay is scaled-up to high throughput screening. Briefly, the assay is conducted in microtitre plates. Following the incubation of cells, test compounds and ³H-labeled PGD₂, the cells are washed and collected onto a Whatman GF/C filter, using a Brandell™ cell harvester. Filters are washed and radioactivity bound to the filters is counted in a beta-counter.

b. Cell Membrane Assay

Radioligand binding assays are performed in 10 mM HEPES/KOH pH 7.4, 1 mM EDTA containing 10 mM MnCl₂ and 0.8 nM [³H]PGD₂, in a final volume of 0.2 ml. The reaction is initiated by the addition of 250 μg of membrane protein, prepared as described above. Ligands in dimethylsulfoxide which is kept constant at 1% (v/v) of the final incubation volume are added. Non-specific binding is determined in the presence of 10 μM PGD₂. Incubation is conducted for 60 min. at room temperature and is terminated by rapid filtration through GF/C filters prewetted in cold assay incubation buffer (no EDTA) using a Brandel™ harvester. The filter is then washed with 4 ml of the same buffer and residual radioligand bound to the filter is determined by liquid scintillation counting following equilibration in 5 ml Ultima Gold™ (Packard).

### EXAMPLE 5

Functional Studies: c-AMP

a. The CRTH2 Receptor

Cells treated as described above are grown to approximately 0.8-1×10⁶ cells/ml on the day of the assay. The cells are harvested by centrifugation at 300 g for 6 min at room temperature, washed with an equal volume of Hanks' balanced salt solution containing 25 mM HEPES pH 7.4 (HBSS/HEPES), re-centrifuged and suspended in HBSS/HEPES. To determine the amount of cAMP cell content, the cells are suspended in HBSS/HEPES containing the adenylate cyclase activator forskolin, which is used to raise intracellular levels of cAMP and the phosphodiesterase inhibitor, 3-isobutyl-1-methylxanthine (IBMX) used to prevent cAMP degradation.

To determine the agonistic activity of a compound, the compound is added to the incubation mixture and the reaction initiated by the addition of the cells and left to proceed for 5-30 min at 37° C. The reaction is stopped by a 3 min incubation in a boiling water bath.

To assess the antagonistic activity of a compound, the cells are pre-incubated for 5-30 min at 37° C. with increasing concentration of the compound. At the end of the pre-incubation, a fixed concentration (EC₅₀ concentration) of the agonist 13,14-dihydro-15-keto-PGD₂ or PGD₂ is added followed by an incubation of 5-30 min at 37° C. The reaction is stopped by a 3 min incubation in a boiling water bath.

b. The DP Receptor

Functional studies for the DP receptor are conducted as described for the CRTH2 receptor except that the assay is conducted in the absence of forskolin.

c. Determination of cAMP

The samples obtained from Example 5 are centrifuged and the cAMP content in the supernatant is determined using [¹²⁵I]-cAMP scintillation proximity assay (Amersham). Maximal inhibition of forskolin stimulated cAMP production by activation of CRTH2 and maximal stimulation of cAMP production due to stimulation of DP are determined in the presence of 10 μM 13,14-dihydro-15-keto-PGD₂ or BW245C, respectively. All compounds are prepared in dimethylsulfoxide (obtained from J. T. Baker, Phillipsburg, N.J.) which is kept constant at 1% (v/v) of the final incubation volume.

### EXAMPLE 6

Functional Studies: Calcium Mobilization

The ability of PGD₂ to elicit a calcium signal in cells expressing a PGD₂ receptor is monitored by measuring the increased fluorescence of the calcium sensing dyes, fura-2 or fluo-3 (Molecular Probe). The cells are first loaded with the calcium sensing fluorogenic dye. After washing away excess dye, the cells are challenged with a PGD₂ receptor agonist to elicit the release of intracellular calcium. Released intracellular calcium bind the fluorogenic dye. Following exposure of the cells to light of the excitation wavelength of the fluorogenic dye, an increase in fluore'scence occurs at the emission wavelength of the fluorogenic dye.

### EXAMPLE 7

Functional Studies: Microphysiometric Assay

Activation of a wide variety of secondary messenger systems result in extrusion of small amounts of acidic metabolites from a cell. The acid formed is largely as a result of the increased metabolic activity required to fuel the intracellular signaling process. The pH changes in the media surrounding the cell are detectable by the CYTOSENSOR™ microphysiometer (Molecular Devices Ltd., Menlo Park, Calif.).

### EXAMPLE 8

Summary of Data

a. HL-60 Cell Line

The results shown in FIG. 1 demonstrate that in the PGD₂ binding assay, the ³HPGD₂ receptor binding ligand does not bind to HL-60 membranes. However, by treating the cells with butyric acid for 7 days, thereby inducing the differentiation of HL-60 cells, the cells acquired the capacity to bind ³HPGD₂.

The results shown in FIG. 2 demonstrate that the binding of tritiated PGD₂ on membranes obtained from differentiated HL-60 cells is only partially (about 10%) competed away by the unlabeled DP-selective ligand BW245C. Using the unlabeled selective ligand for CRTH2, DK-PGD2, about 90% of the tritiated PGD₂ binding on membranes is competed away. The results suggest that there is an increase in the expression of the PGD₂ receptors DP and CRTH2 at the surface of differentiated HL-60 cells. As such, this cell line is useful for screening for compounds capable of binding and/or modulating DP and CRTH2.

b. AML14.3D10 Cell Line

The results shown in FIG. 1 demonstrate that in the PGD₂ binding assay, the ³HPGD₂ receptor binding ligand shows a significant level of binding of PGD₂ to AML14.3D10 membranes. Following the treatment of AML14.3D10 cells with butyric acid, there is increased binding of PGD₂.

Similarly, the results shown in FIG. 2 demonstrate that the binding of tritiated PGD₂ on membranes obtained from nonifferentiated or differentiated AML14.3D10 is only about 10% competed away by the unlabeled DP-selective ligand BW245C. Using the unlabeled selective ligand for CRTH2, DK-PGD2, about 90% of the tritiated PGD₂ binding on membranes is competed away. This suggests that non-differentiated and differentiated AML14.3D10 cells express DP and CRTH2 at their surface. As such, this cell line is useful for screening for compounds capable of binding and/or modulating DP and CRTH2.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention as defined in the appended claims.

What is claimed:

1. A method of screening compounds for binding to a prostaglandin D₂ receptor (PGD₂) comprising:
(a) contacting a host cell expressing said PGD₂ receptor on the surface thereof, or to cell membranes containing said receptor with a labeled compound to be tested for binding affinity to the PGD₂ receptor, wherein said receptor is endogenous to said cell; and
(b) measuring the amount of label bound to the cell membranes of said cells or to said receptor, wherein an increased amount of the label associated with the cell membranes or said host cells indicates that the compound binds to the PGD₂ receptor (FIGS. 1 and 2)

2. The method according to claim 1, wherein said the cells are AML14.3D10 cells or HL-60 cells.

3. The method according to claim 2, wherein said AML14.3D10 cells are undifferentiated.

4. The method according to claim 2, wherein said AML14.3D10 cells are differentiated.

5. The method according to claim 2, wherein said HL-60 cells are differentiated.

6. The method according to claim 1, wherein said PGD₂ receptor is human PGD₂ receptor.

7. The method according to claim 6, wherein the human PGD₂ receptor is one of a DP receptor subtype or a CRTH₂ receptor subtype.

8. An assay for identifying compounds which modulate the activity of a PGD₂ receptor comprising:
(a) contacting cells expressing an endogenous PGD₂ receptor with a compound to be tested for its ability to modulate the activity of said receptor under conditions appropriate for binding to said receptor; and
(b) subsequently monitoring said cells for a resulting change in second messenger activity;
wherein a difference in the second messenger activity of the cell in the presence compared to the absence of said compound indicates that the compound modulates activity of the receptor.

9. A method of inducing cell differentiation in a sample of cells characterized as expressing an endogenous prostaglandin D₂ (PGD₂) receptor, said method comprising contacting said cells with an effective amount of an inducing agent capable of inducing cell differentiation in said cells at a first time point wherein an increased expression of said prostaglandin D₂ (PGD₂) receptor relative to untreated cells is indicative of cell differentiation, and wherein the inducing agent is selected form the group consisting of butyric acid, dimethyl sulfoxide, IL-5, retinoic acid, dibutyryl cyclic-AMP and 5-bromodeoxyuridine.

10. The method according to claim 9, wherein the cell is a cell line selected from the group consisting of AML14.3D10 and HL-60.

11. The method according to claim 9, wherein the PGD₂ receptor is selected from CRTH2 and DP.

12. The method according to claim 9, wherein the agent is butyric acid.

13. The method according to claim 9, further comprising contacting said cells with a second inducing agent at a second time point, wherein said inducing agent is selected form the group consisting of dimethyl sulfoxide, IL-5, retinoic acid, dibutyryl cyclic-AMP and 5-bromodeoxyuridine.

14. The method according to claim 13, wherein the second agent is IL-5.

15. A method of screening for a test compound capable of modulating prostaglandin D₂ receptor activity comprising:
(a) contacting a cell expressing an endogenous PGD₂-specific receptor with a test compound in the presence or absence of PGD₂ or a PGD₂ analogue, and
(b) measuring an effect of the test compound on either an intracellular second messenger or chemokinesis as a measure of the ability of the test compound to modulate PGD₂-specific receptor activity.

16. The method according to claim 15, wherein the cell is a cell line selected from the group consisting of AML14.3D10 and HL-60.

17. The method according to claim 16, wherein the AML14.3D10 and HL-60 cell lines are treated with an inducing agent under conditions favoring increased expression of said PGD₂ receptors just prior to exposure to said labeled PGD₂ or PGD₂ analogue, wherein said inducing agent is selected from butyric acid, dimethyl sulfoxide, IL-5, retinoic acid, dibutyryl cyclic adenosine monophosphate and 5-bromodeoxyuridine.

18. The method according to claim 17, wherein the agent used to induce differentiation is butyric acid.

19. The method according to claim 15, wherein the PGD₂ receptor is one of DP or CRTH2.

20. The method according to claim 15, wherein the intracellular second messenger is selected from the group consisting of cAMP accumulation, inositol phosphate release and calcium ion mobilization.

21. The method according to claim 20, wherein the intracellular second messenger is cAMP.

22. A method of screening for a test compound for binding to a prostaglandin D₂ receptor (PGD₂) comprising:
(a) incubating a host cell expressing an endogenous PGD₂ receptor on the surface thereof, or to cell membranes containing said receptor with one of a labeled PGD₂ or PGD₂ analogue under conditions favoring binding of the labeled PGD₂ or PGD₂ analogue to said receptor;
(b) contacting the mixture from step (a) with a test compound to be tested for binding affinity to the PGD₂ receptor;
(c) measuring the amount of labeled PGD₂ or PGD₂ analogue bound to the cell membranes of said cells or to said receptor as a measure of the ability of the test compound to bind to said receptor, wherein a decreased amount of the label associated with the cell membranes or said host cells indicates that the test compound binds to the PGD₂ receptor.

23. The method according to claim 22, wherein the cell is a cell line selected from AML14.3D10 and HL-60.

24. The method according to claim 23, wherein the AML14.3D10 cell line is undifferentiated.

25. The method according to claim 22, wherein the AML14.3D10 and HL-60 cell lines are treated with a an inducing agent under conditions favoring increased expression of said PGD₂ receptors prior to being contacted with said labeled PGD₂ or analogue, wherein said inducing agent is selected from the group consisting of butyric acid, dimethyl sulfoxide, IL-5, retinoic acid, dibutyryl cyclic adenosine monophosphate and 5-bromodeoxyuridine.

26. The method according to claim 25, wherein the agent is butyric acid.

27. The method according to claim 22, further comprising contacting said cells with a second inducing agent at a second time point, prior to being contacted with said labeled PGD₂ or analogue wherein said inducing agent is selected from the group consisting of butyric acid and IL-5. (Are the cells contacted with inducing agent prior to contact with the labeled ligand or analogue or prior to contact with test compound—the latter would not make sense. Please clarify on a separate piece of paper).

28. The method according to claim 27, wherein the second agent is IL-5.

29. The method according to claim 27, wherein the second agent is butyric acid.

30. The method according to claim 22, wherein the AML14.3D10 and HL-60 cell lines are differentiated.

31. The method according to claim 22, wherein the PGD₂ receptor is selected from the group consisting of DP and CRTH_{2.}

32. A method of determining the ability of a compound to inhibit ligand binding to a prostaglandin D₂ type (PGD₂) cell surface receptor, comprising:
(a) incubating a host cell expressing an endogenous PGD₂ receptor on the surface thereof or to cell membranes containing said receptor with a labeled ligand having binding affinity for the PGD₂ receptor and a test compound; and
(b) determining the level of binding of the ligand to the PGD₂ receptor in the presence of the compound, wherein a lower level of ligand binding in the presence of the compound indicates that the compound binds to the PGD₂ receptor.

33. The method according to claim 32, wherein the cell is a cell line selected from AML14.3D10 and HL-60.

34. The method according to claim 33, wherein the AML14.3D10 cell line is undifferentiated.

35. The method according to claim 34, wherein the AML14.3D10 and HL-60 cell lines are treated with an inducing agent at a first time point sufficient to induce increased expression of PGD₂ receptors relative to untreated cells prior to being incubated with one of said labeled PGD₂ or PGD₂ analogue, wherein said inducing agent is selected from the group consisting of butyric acid, dimethyl sulfoxide, IL-5, retinoic acid, dibutyryl cyclic adenosine monophosphate and 5-bromodeoxyuridine.

36. The method according to claim 35, wherein the inducing is butyric acid.

37. The method according to claim 32, wherein said cells are contacted with a second inducing agent at a second time point, said inducing agent being selected from the group consisting of butyric acid and IL-5. (Is the second contact made after the cells have been contacted with the labeled ligand or just before-please clarify on a separate piece of paper).

38. The method according to claim 37, wherein the second agent is IL-5.

39. The method according to claim 38, wherein the AML14.3D10 and HL-60 cell lines are differentiated (This is redundant considering that the cells have already been induced to differentiate).

40. The method according to claim 32, wherein the PGD₂ receptor is one of DP or CRTH₂.

41. A compound identified by any one of the preceding claims.

42. A method for screening and identifying antagonists of a human prostaglandin D₂ moiety, comprising:
(a) contacting a cell line that expresses a prostaglandin D type cell receptor with a test compound in the presence of the prostaglandin moiety; and
(b) determining whether the test compound inhibits the binding and cellular effects of the prostaglandin moiety on the cell line, in which antagonists are identified as those compounds that inhibit both the binding and cellular effects of the prostaglandin moiety on the cell line.

43. The method according to claim 42, wherein said moiety is PGD₂.

44. A method for identifying a ligand(s) that activates a prostaglandin D₂ type cell receptor, the method comprising:
(a) challenging host cells that endogenously expresses the PGD₂ cell receptor with candidate ligand(s) which can potentially bind with the ligand-binding domain of the PGD₂ type cell receptor, wherein the cells also contain a reporter gene functionally linked to a hormone response element responsive to the reporter gene; and
(b) monitoring induction of the reporter gene(s), thereby identifying ligand(s) that activate the PGD₂ type cell receptor.

45. A method for identifying agonist or antagonist of a human prostaglandin D₂ moiety comprising:
(a) contacting cells expressing on the surface thereof a prostaglandin D type cell receptor, wherein the receptor is associated with a second component capable of providing a detectable signal in response to the binding of a compound to the receptor, with a compound to be screened under conditions favoring binding of the compound to the cell surface receptor; and
(b) determining whether the compound binds to and activates or inhibits the cell surface receptor by measuring the level of a signal generated from the interaction of the compound with the cell surface receptor protein.

46. A method for screening and identifying agonists of human prostaglandin D₂ type moiety or metabolite, comprising:
(a) incubating or exposing a cell line that endogenously expresses a human prostaglandin D₂ type cell surface receptor with the human prostaglandin D₂ type moiety or metabolite in the presence and in the absence of the test compound having binding affinity for the cell surface receptor;
(b) determining whether, the presence of the test compound reduces the binding of the prostaglandin D₂ type moiety or metabolite to the cell surface receptor in the cell line relative to a control, and
(c) determining whether, in the absence of the prostaglandin moiety, the test compound mimics the cellular effects of the moiety on the cell line, in which agonists are identified as those test compounds that inhibit the binding but mimic the cellular effects of prostaglandin moiety on the cell line.
